(19) United States
(12) Patent Application Publication
(10) Pub. No.: US 2012/0304992 A1
(43) Pub. Date: Dec. 6, 2012

(54) Breathing treatment system and method

(76) Inventors: David R. Ratto, Arcadia, CA (US); Richard L. Murray, Upland, CA (US)

(21) Appl. No.: 13/134,315

(22) Filed: Jun. 6, 2011

(51) Int. Cl. A61M 16/16; A61M 16/00

(52) U.S. Cl. 128/203.26; 128/207.18

(57) ABSTRACT

A cannula tip for insertion in a patient's nostril includes nostril- and cannula-ends, with a cannula-end that has a greater cross section than that of the nostril end. The cross section of the cannula tip may decrease from its cannula end to its nostril end linearly or non-linearly.

### CROSS REFERENCE TO RELATED APPLICATION

This application is related to and claims benefit of the filing date of application Ser. No. 12/804,612, having the same inventors as the present application, entitled, “BREATHING TREATMENT SYSTEM AND METHOD,” filed Jul. 26, 2010, and Ser. No. 12/924,762, having the same inventors as the present application, entitled, “BREATHING TREATMENT SYSTEM AND METHOD,” filed Oct. 5, 2010, which are hereby incorporated by reference.

### FIELD

Disclosed subject matter is related to assisted-breathing systems and methods.

### BACKGROUND

Nasal cannulas are widely used to treat patients for a variety of conditions. Cannulas provide a conduit for respiratory gases between a respiratory gas source and a patient's respiratory system via the patient's nostrils. Such respiratory gases may be required for critical care, such as oxygen therapy in emergency room situations, or for continuing care in hospital or out-patient settings, for example. Nasal cannulas supply respiratory gases relatively conveniently and at low cost, yet, even cannulas that may otherwise be adequate for their intended purposes, may exhibit undesirable characteristics. For example, cannulas may generate or amplify distracting or annoying levels of noise in operation. Such noise can be particularly problematic in a setting such as a hospital, where patients are already under stress and in need of rest. In other environments, such as out-patient or in-home care environments, noise generated by the delivery of respiratory gases through cannulas may disturb not only patients receiving respiratory treatment, but other patients or members of a patient's household, for example. An improved nasal cannula would therefore be highly desirable.

### SUMMARY

In an apparatus and method in accordance with the principles of the present invention, a cannula tip (also referred to as a cannula prong) features openings at a cannula-end and at a nostril-end. The cannula tip's cannula end may be integral to or coupled to a cannula. The cannula tip's nostril end is situated at the opposite end from the cannula end and is configured for insertion in a patient's nostril. In accordance with the principles of the present invention, the nostril end of the cannula tip may be characterized by a smaller cross-section than the cross-section of the cannula-end. Not wishing to be bound by theory, it is believed that a cannula tip having a smaller opening at its nostril end than at its cannula-end may establish vortex action, thereby allowing greater flow rates for respiratory gases and reduced noise levels.

In illustrative embodiment, a cannula tip may taper smoothly from its cannula-end to its nostril-end. That is, in such an illustrative embodiment, the decrease in inside diameter of the cannula-tip is linear. In other embodiments, the decrease in inside diameter of the cannula tip may be nonlinear, characterized by an elliptic or hyperbolic curve, or with a plurality of curved segments such as conical and cylindrical segments, for example. Other nonlinear reductions in cross-section are contemplated within the scope of claimed subject matter.

In another aspect, a cannula tip in accordance with the principles of the present invention may include a nostril end that is either occluding or non-occluding (i.e. restricts gas flow to the interior of the cannula itself, or allows gas flow outside the cannula in the region between the cannula and a patient's nostril). In another aspect, a nostril tip in accordance with the principles of present invention may be integral to an associated cannula, or, in replaceable embodiments, a cannula tip in accordance with the principles of the present invention may be configured for detachable connection to an associated cannula, thereby permitting ready-replacement of the tip to enhance good sanitary practices. An easy replacement embodiment may for example, include mating snap-fit structures on the cannula and cannula end of the cannula tip, “slip on” mating connection, or threaded mating ends, for example.

A nasal cannula in accordance with the principles of present invention may include a nostril tip or mating structure for receiving a nostril tip having a greater cross-section its cannula and then at its nostril end. A cannula in accordance with the principles of the present invention may be of a the single-channel or dual-channel configuration. In accordance with the principles of present invention, a single-channel cannula may include a source end associated with a respiratory gas supply, and a nostril end positioned within a patient's nostrils. Nostril tips, proximate the nostril end direct respiratory gas received from the source end into a patient's nostrils. A multi-channel cannula, on the other hand, may include two open ends with nostril tips located between the two open ends of such a cannula. The two open ends of such a cannula may coupled to a respiratory gas source to provide dual pathways for delivery of gases to nostril tips.

Nostril tips in accordance with the principles of the present invention, and nasal cannulas employing the same may be used in a variety of respiratory gas delivery systems, including an open-delivery (that is, non-occluding) system that delivers high flow rate humidified respiratory gas to a patient.

In high flow-rate applications, including non-occluding high flow-rate applications, a nostril tip in accordance with the principles of the present invention may substantially reduce the noise associated with the delivery of respiratory gases by virtue of its tapered shape (that is, a nostril-tip having a greater opening at its cannula-end than at its nostril-end). Additionally, a nostril tip in accordance with the principles of claimed subject matter may include materials that render the tip somewhat more rigid than many conventional nostril tips. Such materials may include relatively rigid formulations of ABS plastic, polypropolene, polyvinylchloride, polycarbonate, or polystyrene, for example. Nostril tips in accordance with the principles of claims subject matter may be of a variety of sizes, but, in any case, are sized to allow ready flow of respiratory gases, particularly in high-flow rate applications.

A nostril tip in accordance with the principles of the present invention may be particularly well-suited to operation with a high flow-rate cannula for use in high flow rate respiratory therapy. Such a cannula may have an inside diameter in a range from 6.0 mm to 15.0 mm, the inside diameter of the cannula end of a nostril tip may range from 5.0 mm to 14.0 mm, and the inside diameter of the nostril end of a nostril tip may range from 3.5 mm to 13 mm, for example. A cannula for use in high flow rate applications may, in addition to having a larger inside diameter than conventional cannulas, have a relatively smooth inner surface (e.g., no corrugation), allow for only smooth direction changes (e.g., no turns of greater than sixty degrees), and smooth transitions (e.g., any transition, at a joint, such as a “slip on” connection, for example, greater than twenty percent of the inside diameter of the cannula may be distributed along the cannula for a length at least equal to the diameter of the cannula).

### BRIEF DESCRIPTION OF THE FIGURES

Non-limiting and non-exhaustive embodiments will be described with reference to the following Figures, wherein like reference numerals refer to like parts throughout the various Figures unless otherwise specified.

FIGS. 1A through 1C are perspective views of a cannula tip in accordance with the principles of the present invention;

FIGS. 2A through 2C are sectional views of detachable and integral cannula tips in accordance with the principles of the present invention;

FIGS. 3A and 3B are plan views of cannula ends including cannula tips in accordance with the principles of the present invention;

FIGS. 4A and 4B are plan views of illustrative embodiments of respiratory air cannulas in accordance with the principles of the present invention;

FIG. 5 is a block diagram of an illustrative embodiment of a respiratory gas supply system such as may employ a cannula tip in accordance with the principles of the present invention.

### DETAILED DESCRIPTION

Although claimed subject matter will be described in terms of certain embodiments, other embodiments, including embodiments that do not provide all of the benefits and features set forth herein, are also within the scope of this invention. Various structural, logical, process step, and electronic changes may be made without departing from the spirit or scope of the invention. Flow charts may include steps that may be deleted or otherwise modified and the sequence set forth within a particular flow chart may be modified while keeping within the scope of the invention. Accordingly, the scope of the invention is defined only by reference to the appended claims.

In an apparatus and method in accordance with the principles of the present invention, a cannula tip features openings at a cannula-end and a nostril-end. The cannula tip's cannula end may be integral to or coupled to a cannula. The cannula tip's nostril end is situated at the opposite end from the cannula end and is configured for insertion in a patient's nostril. In accordance with the principles of the present invention, the nostril end of the cannula tip may be characterized by a smaller cross-section than the cross-section of the cannula-end.

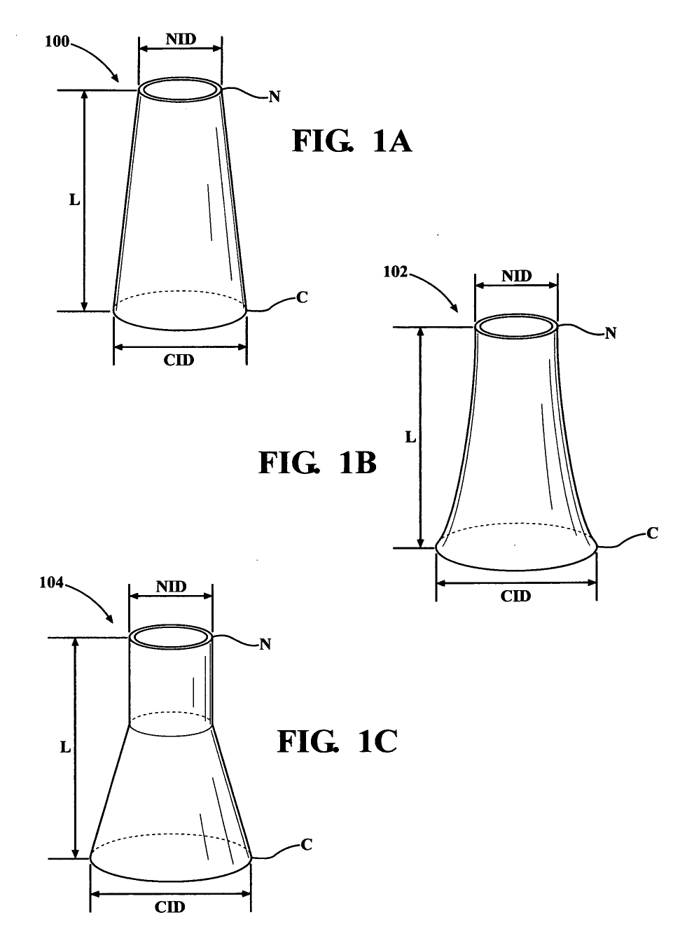

In the illustrative embodiment of FIG. 1 a cannula tip 100 is formed as a tube with open cannula and nostril ends, C and N, respectively. The cannula end C of the cannula tip 100 receives respiratory gas from a respiratory gas source and the respiratory gas flows through the cannula tip 100 to the nostril tip end N. In operation, the nostril tip end N is inserted in a patient's nostril for treatment. Respiratory gas is thereby routed from a respiratory gas source, through a cannula and cannula tip 100 to patient's nostril, and, from there, introduced to a patient's respiratory system. A cannula tip in accordance with the principles of the present invention may employ a flexible material, such as a soft medical-grade vinyl, for example, or, in high flow-rate applications, including non-occluding high flow-rate applications, a nostril tip in accordance with the principles of the present invention may include materials that render the tip somewhat more rigid than many conventional nostril tips. Such materials may include relatively rigid formulations of ABS plastic, polypropolene, polyvinylchloride, polycarbonate, or polystyrene, for example. A nostril tip in accordance with the principles of the present invention may substantially reduce the noise associated with the delivery of respiratory gases by virtue of its tapered (that is, with a nostril opening having a lesser cross-section than its cannula end) shape.

Nostril tips in accordance with the principles of claims subject matter may be of a variety of sizes, but, in any case, are sized to allow ready flow of respiratory gases, particularly in high-flow rate applications. Proper sizing of nostril-tip openings may be determined empirically for different flow rates, cannula sizes, and nostril openings, for example.

In illustrative embodiments, the inside diameter CID of the cannula end C of the cannula tip 100 may range from 3.0 mm to 15 mm, for example. The inside diameter CID may be substantially equal, for example, to the diameter of a cannula with which the tip 100 is combined. The inside diameter NID of the nostril end N of the cannula tip 100 may range from 2.5 mm to 10 mm, but less than the inside diameter CID of the cannula end C. That is, if the cannula end inside diameter CID is 15 mm, the nostril end inside diameter NID may range up to, but less than, 15 mm, for example. In another aspect, a cannula tip in accordance with the principles of the present invention include a nostril end that is either occluding or non-occluding. In illustrative embodiments in which respiratory gas is to be supplied to a patient at relatively high flow rates (e.g., twelve to eighty liters per minute), the cannula tip 100 may be sized to accommodate high flow rates without impeding flow. In an illustrative embodiment of such a high flow rate cannula, the inside diameter of the cannula may range from 6.0 mm to 8.0 mm, the inside diameter CID of the cannula end C may range from 5.0 mm to 7.0 mm, and the inside diameter NID of the nostril end N may range from 3.5 mm to 6 mm, for example. Generally, cannulas of larger cross section may accommodate higher flow rates and cannulas of smaller cross section may accommodate lower flow rates, without impeding flow. In an illustrative embodiment of higher flow rate cannulas, the inside diameter of the cannula may range from 6.0 mm to 15.0 mm, the inside diameter CID of the cannula end C may range from 5.0 mm to 14.0 mm, and the inside diameter NID of the nostril end N may range from 3.5 mm to 13 mm, for example.

The length L of the cannula tip 100 may range from 5.0 mm to 40 mm. Different lengths may be suitable for different patients. That is, because patient's noses vary in dimension, the length L that provides comfort and utility may vary accordingly. In an illustrative high flow rate embodiment the length L is between 12 mm and 18 mm. In the illustrative embodiment of FIG. 1A the inside diameter of the tip 100 diminishes linearly from cannula end C to nostril end N, adopting a cone shape.

In other embodiments, the decrease in inside diameter of the cannula tip may be nonlinear, characterized by an elliptic or hyperbolic curve, or with a plurality of curved segments such as conical and cylindrical segments, for example. Other nonlinear reductions in cross-section are contemplated within the scope of claimed subject matter. In the illustrative embodiment of FIG. 1B, for example, a cannula tip 102 decreases in cross section nonlinearly from a cannula end inside diameter CID to nostril end inside diameter NID, with the sides of the tip 102 exhibiting a generally elliptic curve. The length of the cannula tip 102 may be as described in the discussion related to FIG. 1A, for example.

In the illustrative embodiment of FIG. 1C nostril tip 104 may be characterized, in part, by a nonlinear decrease of the inside diameter from cannula end to nostril end that exhibits two distinct regions: a conical region 106 and a cylindrical region 108. Nostril tip 104 may include embodiments in which the nostril end inside diameter NID and cannula end inside diameter CID and length L, may fall into ranges as described in the discussions later to figures for 1a and 1B, for example, with a nostril end inside diameter NID less than the cannula end inside diameter CID.

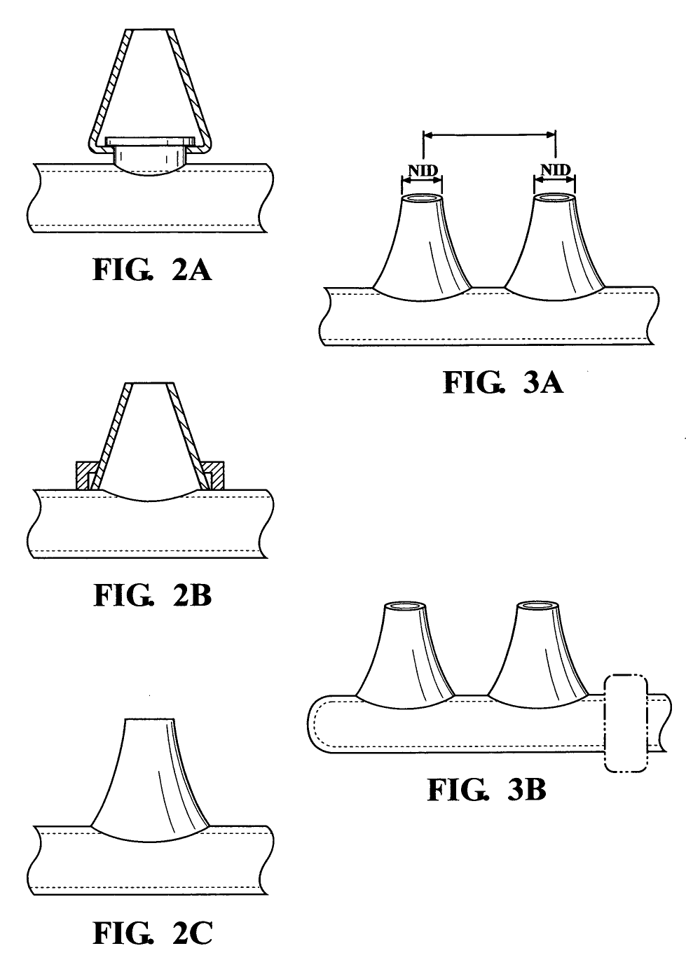

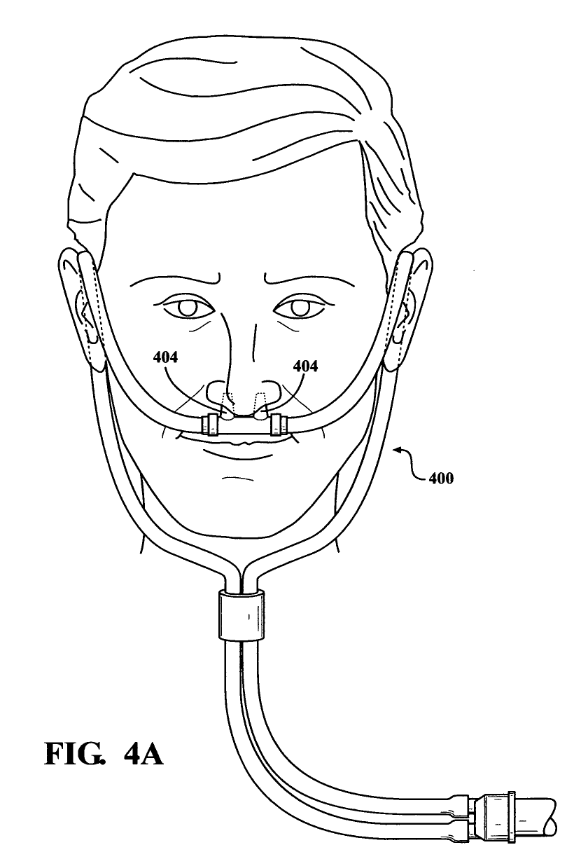

As will be described in greater detail in the discussion related to FIGS. 2A through 2C, a nostril tip in accordance with the principles of present invention, such as those described in the discussion related to FIGS. 1A through 1C may be integrated with a cannula or maybe detachable from a cannula. Detachable embodiments allow for frequent, relatively expensive replacement of tips, thereby encouraging good sanitary practice. That is, although widely recommended, patients often neglect to clean and/or replace respiratory equipment. Employing detachable nostril tips increases the probability that patients will frequently replace respiratory system components most likely to harbor agents of disease and infection. In replaceable embodiments, a cannula tip in accordance with the principles of the present invention may be configured for detachable connection to an associated cannula, thereby permitting ready-replacement of the tip to enhance good sanitary practices. An easy replacement embodiment may for example, include mating snap-fit structures on the cannula and cannula end of the cannula tip, or threaded ends, for example. As will be described in greater detail in the discussion related to FIG. 3B a cannula end may also be detachable in accordance with the principles of the present invention.

In the illustrative embodiment of FIG. 2A the cannula end C of the cannula tip 200 may be press-fit around a nipple 202 formed in a cannula 204. In this illustrative embodiment the inside diameter of the nipple 202 may be the inside diameter of the cannula tip. That is, the decrease in cross-section from cannula end to nostril end of the tip 200 may be measured starting at the outlet of the nipple 202. In the illustrative embodiment of FIG. 2B a cannula tip 203 may be formed to make mating engagement with a cannula 204 by press fitting inside a nipple 206 that includes a rim 208 that engages with a cannula tip inserted into the nipple 206. The rim 208 may be formed, for example, of a flexible material to permit relatively easy insertion of the cannula end of the tip 204. As previously indicated, a more rigid material may be employed in high flow-rate applications. In an illustrative embodiment of FIG. 2c a cannula tip 210 may be integral to a cannula 212, formed at the same time, and of a piece with, the cannula 212, for example.

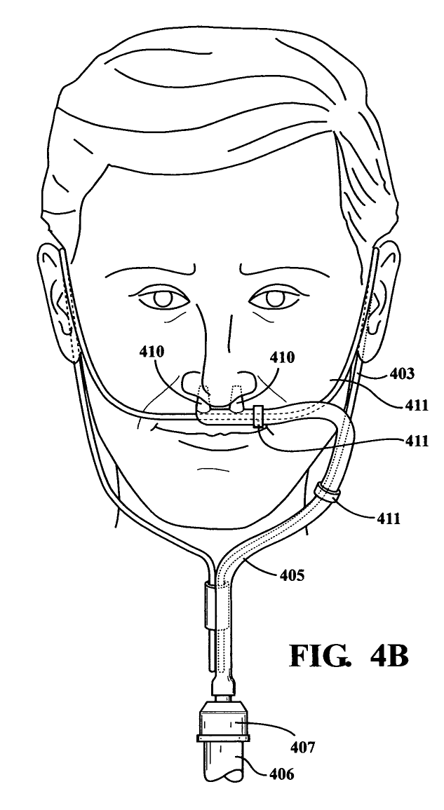

In the illustrative embodiment of FIG. 3A a nasal cannula 300 includes spaced-apart nostril tips 302,304 in accordance with principles of the present invention. The tips 302,304 may be integral to the cannula 300 or may be detachable, for example. The length L, nostril end inside diameter NID, and cannula end inside diameter CID may fall within ranges as described in discussions related to other figures herein, with a nasal end inside diameter less than the cannula end the inside diameter. The center-to-center spacing of tips 302, 304 may vary according to patient requirements, but, generally, it will fall in the range of 5 mm to 30 mm. In this illustrative embodiment, the tips 302, 304 are positioned along the cannula 300 between two open ends that are coupled to a respiratory gas supply. A cannula 300 having two open ends according to this embodiment may be draped around a patient's ears for support, for example, and will be described in greater detail in the discussion related to FIG. 4A.

In an illustrative embodiment of FIG. 3B a single-ended cannula 306 includes spaced-apart nostril tips 308,310 situated proximate the terminal, or nostril, end of cannula 306. Nostril tips 308,310 may be integral to cannula 306 or detachable, for example. The center-to-center and spacing of nostril tips 308,310 may vary to accommodate different patients, but generally, will fall in the range of between 5 mm and 30 mm. The length L, nostril end inside diameter NID, and cannula end inside diameter CID may be as described in the discussion related to FIGS. 1A through 1C, for example. In this illustrative embodiment, cannula 306 provides a single channel for flow of respiratory gas from a respiratory gas source to nostril tips 308, 310. As will be described in greater detail in the discussion related to FIG. 4B, a single-ended cannula in accordance with this embodiment may provide greater comfort and convenience for patient than a cannula that supplies respiratory gas from two channels draped around a patient's head, for example. In an illustrative embodiment, a cannula in accordance with the principles of the present invention may include a detachable end piece 312 which may be conveniently replaced to maintain sanitary standards without removing and replacing an entire cannula. In an illustrative embodiment a joint 314, which may be press-fit or a threaded joint, for example, may be included for mating attachment of the cannula end piece 312 to a tube 316 of the cannula 306.

A nasal cannula in accordance with the principles of present invention may include a nostril tip, or mating structure for receiving a nostril tip, having a greater cross-section at its cannula end than at its nostril end. A cannula in accordance with the principles of the present invention may be of a single-channel or dual-channel configuration. In accordance with the principles of present invention, a single-channel cannula may include a source end associated with a respiratory gas supply, and a nostril end positioned within a patient's nostrils. Nostril tips, proximate the nostril end, direct respiratory gas received from the source end into a patient's nostrils. A multi-channel cannula, on the other hand, may include two open ends with nostril tips located between the two open ends of such a cannula. The two open ends of such a cannula may coupled to a respiratory gas source to provide dual pathways for delivery of gases to nostril tips.

In an illustrative embodiment of FIG. 4A, a cannula 400 includes a mating connector 402 for connection to a conduit such as conduit 306 of FIG. 3A. In this illustrative embodiment diminished cross section nasal tips 404 in accordance with the principles of the present invention may be integral to or detachable from a tube 403 that provides dual-channel delivery of respiratory gas to a patient. Connector 402, which may be separately formed and friction-attachable to flexible tubing that forms cannula 400, may allow the cannula 400 to swivel relative to the conduit 306 and to thereby accommodate a patient's motion, for example. The total length of the cannula may vary from patient to patient, but will typically be long enough to allow the cannula to be draped over patient's ears for support. In illustrative embodiments, the length of the cannula 400 varies from one-one third to two meters.

The inside diameter of the cannula 400 may be such that the cross-section is half the cross-section of the conduit to which the cannula is attached, in an embodiment in which a cannula “splits” to provide two flow paths which, for example, may be draped from a patient's ears and deliver respiratory gas from both sides of a patient's head. Cannula 400 may include flexible tubing material and, in an illustrative embodiment, has an inside diameter, D, of between 3 mm and 15 mm, preferably between 4.5 mm and 9 mm, more preferably between 4.75 mm and 6.0 mm. Tubing that supplies respiratory gas to the cannula 400 may have an inside diameter of between 10 mm and 30 mm twelve, preferably between 10 mm and 20 mm, more preferably between 12 mm and 15 mm in an illustrative embodiment. In this manner, flow is not restricted in the transition from tubing 406 to cannula 400. In illustrative embodiments, cannula 400 and tubing 406 may be formed of one piece or may include a plurality of connected segments. Cannula 400 and tubing 406 may include heating elements and/or insulation to prevent condensation, also referred to herein as “rainout.” Because cannula 400 may have more intimate contact with a patient, it may be advantageous to replace cannula 400 more frequently than tubing 406. Multi-segment embodiments allow cannula 400 to be replaced, while retaining tubing 406. As described in greater detail in the discussion related to FIGS. 1A through 3B nostril tips 404 may be replaceable, allowing ready replacement of those components making most intimate contact with a patient and, therefore, most likely to harbor agents of disease.

In this illustrative embodiment, tips for insertion in a patient's nose are situated approximately midway along the cannula 400. In illustrative embodiments the tips range from 5 mm to 30 mm in length. Inside diameters of the nasal tips 404 are chosen to allow for free flow of respiratory gas and their total cross-section may therefore be of a size to approximate the cross-section of the conduit 406 with which the cannula is connected in this illustrative embodiment, but, as previously described, the nostril end N of each tip is of a lesser cross-section than that of the cannula end C. In an illustrative embodiment the inside diameter of each tip may be between 3 mm and 15 mm, preferably between 4 mm and 6 mm, more preferably between 4.25 mm and 4.75 mm. The cross section of tips 404 may be reduced from the cannula end to the nostril end in order to reduce the noise associated with flowing respiratory gas. Tips may be non-occluding, or occluding, depending, for example, upon the respiratory therapy involved.

Rather than splitting the flow of respiratory gas between two paths, as conventional cannula do, an open nasal cannula in accordance with the principles of claimed subject matter may employ a single path in order to maximize gas flow. In the illustrative embodiment of FIG. 4B an open nasal cannula 401 includes a single conduit 405 having an attachment mechanism 407 at its proximal end configured for attachment to respiratory gas conduit 306. At its distal end, the cannula 401 includes tips 410 for insertion into a patient's nostrils.

Cannula 401 may include flexible tubing material and, in an illustrative embodiment, has an inside diameter, D, of between 3 mm and 15 mm, preferably between 4.5 mm and 9 mm, more preferably between 4.75 mm and 6.0 mm. Tubing that supplies respiratory gas to the cannula 401 may have an inside diameter of between 10 mm and 30 mm, preferably between 10 mm and 20 mm, more preferably between 12 mm and 15 mm in an illustrative embodiment. In this manner, flow is not restricted in the transition from tubing 406 to cannula 401. In illustrative embodiments, cannula 401 and tubing 406 may be formed of one piece or may include a plurality of connected segments. Cannula 401 and tubing 406 may include a heating element and/or insulation to prevent condensation.

A cannula with large inside diameter may permit delivery of respiratory gas at high flow rates while requiring less work of a compressor than would be required with a cannula of smaller cross section. Additionally, a cannula with larger cross section may reduce noise associated with high flow-rate delivery of respiratory gases. In an illustrative embodiment attachment means 411, such as clamps or straps, for example, may be integrated with, or attached to, a cannula 401 in accordance with the principles of claimed subject matter. Attachment means 411 may be used along with a support device 403, such as a lanyard, that may be used to hold the cannula 401 in position to facilitate delivery of respiratory gas to a patient's nostrils. In an illustrative embodiment, support device 403 may be composed of a length of three-millimeter rubber tubing, for example, and may be draped over a patient's ears to support cannula 401. An adjuster 423, which may be a friction-fit device, for example, may be used to adjust snugness of fit of the length of tubing as the tubing loops around or otherwise engages with a patient's head. In such an illustrative embodiment the cannula may be firmly held in place without the discomfiture associated with conventional masks or the tendency of conventional cannulas to move out of place. Additionally, in such an embodiment, one side of the mouth is left relatively open, with no cannula loop obstructing one side of a patient's face. Without the obstruction of a cannula, a patient may find it easier to perform regular tasks, such as eating, drinking, or talking on the telephone, for example. In contrast to conventional cannulae, which loop over a patient's ears, thereby annoying a patient and which, additionally, tend to fall out from behind the patient's ears because they are too bulky, a cannula system in accordance with claimed subject matter, which is supported by a relatively light and slender device, such as tubing 403 and may include an adjustment mechanism 423, may provide much more comfortable and reliable delivery of respiratory gas to a patient.

Nostril tips in accordance with the principles of the present invention, and nasal cannulas employing the same, may be used in a variety of respiratory gas delivery systems, including an open-delivery (that is, non-occluding) system that delivers high flow rate humidified respiratory gas to a patient.

In an apparatus and method in accordance with the principles of claimed subject matter, warm, humidified, respiratory gas may be supplied to a patient through an open delivery system for treatment of respiratory conditions, including obstructive sleep apnea, hypopnea, congestive heart failure, or respiratory failure, for example. Rather than forcing a respiratory gas into a patient's respiratory system by sealing a patient's breathing orifices (e.g., nostrils, and, in some cases, mouth) and forcing a gas under pressure into the patient's nostrils, as a conventional closed system (e.g., CPAP system) would, a system in accordance with the principles of claimed subject matter may supply a respiratory gas to a patient's nostrils through a cannula having open tips configured for insertion in a patient's nostrils.

It is believed that respiratory gas delivered at a high flow rate develops positive airway pressure within a patient's respiratory passage, thereby opening the passage. The cannula does not require a mask, nor does it form a gas-tight seal with the patient's nostrils. As a result, a patient may experience greater comfort and ease of breathing and more readily comply with respiration therapy. Because respiratory gas is supplied from an open delivery system (that is, respiratory gas is allowed to escape from the patient's nostrils) a patient may exhale more easily than with conventional closed delivery systems that don't allow respiratory gas to escape through a patient's nostrils during exhalation. A patient's comfort may be further enhanced in this manner and may be more compliant with his respiration therapy as a result. Additionally, an open system in accordance with the principles of claimed subject matter avoids the creation of painful sores caused by gas escaping through localized gaps between a mask and the patient's skin, as may occur with conventional closed (i.e., purportedly gas-tight) respiratory gas delivery systems.

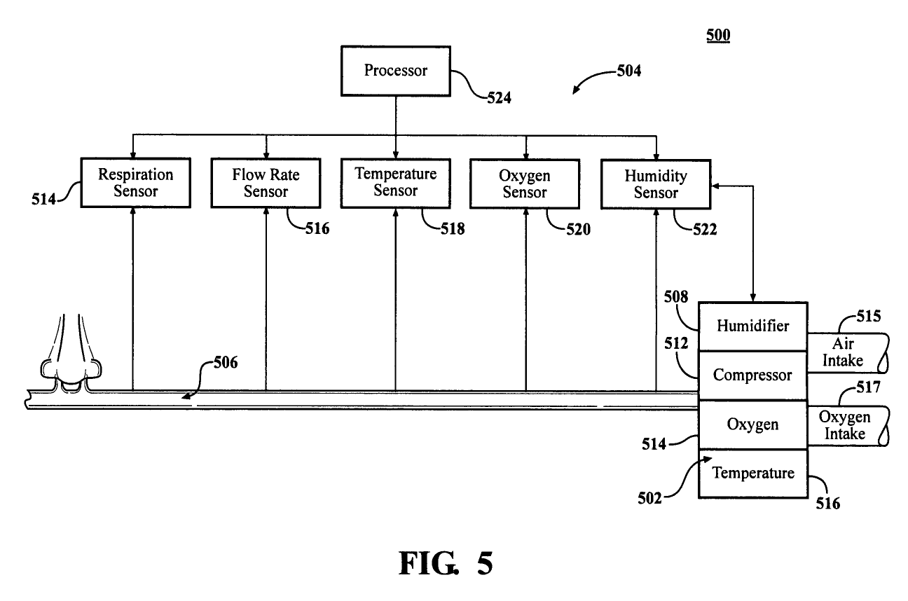

The block diagram of FIG. 5 includes components of an illustrative embodiment of an open delivery respiratory system 500 in accordance with the principles of claimed subject matter. The system 500 includes a respiratory gas conditioner 502, a controller 504, and a delivery component 506. Controller 504 may be implemented using a variety of technologies, including: analog circuitry, digital circuitry, hybrid components, logic arrays, or microprocessor technology, for example. The respiratory gas conditioner 502 may include a humidifier 508, a heater 510, a cooler 511, a compressor 512, or an Oxygen supply system 514. In illustrative embodiments a system 500 in accordance with claimed subject matter may include individual elements (e.g., heater 510, humidifier 508), gas conditioner 502 or combinations thereof. A controller 504 may include a respiration sensor 514, a flow sensor, a temperature sensor 518, an oxygen sensor 520, a humidity sensor 522, or a processor 524, for example. Valves, such as scissor valves, actuators, or servo-valves (not shown) may also be used to control gas flow. Sensors may be situated anywhere within the system and may be used to regulate respective respiratory gas characteristics. In an illustrative embodiment, a flow sensor may be positioned where respiratory gas enters the humidifier and/or where respiratory gas supply tubing meets a nasal cannula, for example. A nasal cannula, including nostril tips as described in the discussions related to previous figures, may be employed to deliver respiratory gas to a patient from the system.

In an illustrative embodiment, a temperature sensor 518 senses the temperature of respiratory gas and provides feedback to heater 510 and cooler 511 in order to regulate the temperature of respiratory gas to a range between 30° C. and 40° C., or in a preferred embodiment, between 34° C. and 39° C., or, in a still more preferred embodiment, between 36° C. and 38° C., at the point of delivery to a patient. A thermistor sensor may be used as temperature sensor 518, for example. As described in greater detail in the discussion related to the following Figures, a respiration sensor 514 may be employed by a respiratory system 500 to detect characteristics of a patient's breathing, such as the initiation of inhalation or exhalation, for example. Respiration sensors are known. A pneumatic belt breathing sensor is described in U.S. Patent No. 4,602,643 issued to Henry G. Deitz, and piezo-electric belt sensors are known and available, for example, from iWorx/CB Sciences, One Washington Street, Suite 404, Dover N.H. 03820, and pyroelectric polymer (PEP) films have been proposed as transducers for respiratory rate monitors, for example. A zRIP respiratory inductance sensor belt may be obtained from Pro-Tech, online, at http://www.pro-tech.com/scripts/asp/prod zrip.asp. Such sensors may detect movement of a patient's chest wall, for example.

Measurements obtained by respiration sensor 514 may be employed by the controller 504 to adjust the rate of flow of respiratory gas supplied by the system 500. For example, an indication from a respiration sensor 514 that a patient has begun to inhale may be used by the controller 504 to increase the flow of respiratory gas, or an indication from a respiration sensor 514 that a patient has begun to exhale may be used by the controller 504 to diminish, or even cut off entirely, the flow of respiratory gas. Respiratory gas flow rates sensors are described in, “Wireless Microsensor System for Monitoring a Breathing Activity”, IFMBE Proceedings, 4^{th} European Conference of the International Federation for Medical and Biological Engineering, by Jos Vander Sloten, Pascal Verdonck, Marc Nyssen, and Jens Hauesien, for example.

A flow sensor 516 may be used to determine the rate of flow of respiratory gas and, when compared with a target value in a feedback configuration, may be used to regulate the flow rate of the respiratory gas. In various illustrative embodiments, the flow rate may be a single, preset, value; may be multi-valued, with, for example, a low flow rate for a predetermined period (e.g., 1.5 seconds) followed by a high flow rate for a predetermined period (e.g., 2.5 seconds); or may be set at a high flow rate when a patient inhales and be set at a low flow rate when a patient exhales, for example. Continuous high frequency oscillation (CHFO), or high frequency oscillatory ventilation (HFOV), may also be employed by a system in accordance with the principles of claimed subject matter. CHFO is known and described, for example, in and article entitled, “High Frequency Oscillatory Ventilation” in “The Internet Journal of Emergency And Intensive Care Medicine 2003, Vol. 6, No. 2,” available at http://www.ispub.com/ostia/index.php?xmlFilePath=journals/ijeicm/vol6n2/hfov.xml. Combinations of such flow rate settings are contemplated within the scope of claimed subject matter. A high flow rate may vary from twelve to eighty liters per minute (LPM), for example. The appropriate flow rate for a given patient may be determined, for example, by a titration process which determines a minimal flow rate required to establish and maintain an open airway for the patient. The operating flow rate may be set at a slightly, higher rate than the minimum rate required to maintain an open airway in order to provide some operating margin (e.g., a margin of two LPM), for example. A low flow rate may vary from zero to ten LPM. Respiratory gas may include air, received from air intake 515 or oxygen received through an oxygen intake 517 from an oxygen supply, such as a commercially available oxygen tank, for example. As described in greater detail below, various combinations of air and oxygen are contemplated within the scope of the claimed subject matter.

In illustrative embodiments a respiratory gas supply system 500 may determine gas supply flow rates by presetting a valve opening. That is, rather than measuring the flow rate of the respiratory gas and providing feedback to a controller 524, a respiratory gas supply system 500 in accordance with the principles of claimed subject matter may have flow rate and valve settings correlated, in a manufacturing or test setting, for example, so that flow rates may be determined by adjusting valves to predetermined settings. In such illustrative embodiments the flow rate may be a single rate, or may be adjustable to a plurality of rates, for example. As with a system that employs flow rate feedback, the flow rate may be set at a high level while a patient inhales and at a low level while the patient exhales, for example. Humidity sensor 522 may be employed in closed-loop feedback regulation of respiratory gas embodiments of a respiratory system 500 in accordance with the principles of claimed subject matter. Humidity sensors are known and described, for example, in U.S. Pat. No. 6,895,803, issued to Seakins et al, and entitled, Humidity Sensor. Oxygen sensor 520 may be used in illustrative closed-loop feedback embodiments of a respiratory gas supply system in accordance with the principles of claimed subject matter to regulate the percentage by volume of oxygen of supplied respiratory gas. Oxygen sensors are known and described, for example in U.S. Pat. No. 4,109,509 issued to Cramer et al and entitled, Oxygen Monitoring and Warning Device for Aircraft Breathing System.

It is to be understood that other embodiments may be used, for example, or changes or alterations, such as structural changes, may be made. All embodiments, changes or alterations, including those described herein, are not departures from scope with respect to intended claimed subject matter. Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments. While there has been illustrated and described what are presently considered to be example embodiments, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular embodiments disclosed, but that such claimed subject matter may also include all embodiments falling within the scope of the appended claims, and equivalents thereof.

1. A tip for delivering respiratory gas to a nostril of a patient, comprising:
a first open end characterized by a first inside diameter, the first end adapted for insertion into the patient's nostril;
a second end characterized by a second inside diameter that is greater than the inside diameter of the first end, the second end adapted for mating engagement with a nasal cannula; and
a conduit between the first and second open ends.

2. The tip of claim 1 wherein the outside diameter of the end having the smaller inside diameter is of a dimension that avoids occlusion of a nostril into which it is to be inserted.

3. The tip of claim 1 wherein the inside diameter of the tip varies from the larger-diameter end to the smaller diameter end linearly.

4. The tip of claim 1 wherein the inside diameter of the tip varies from the larger-diameter end to the smaller diameter end non-linearly.

5. An apparatus for delivering respiratory gas to a patient, comprising:
a nasal cannula for coupling to a respiratory gas supply; and
a nostril tip for insertion into a nostril of the patient, the tip coupled to the nasal cannula to convey respiratory gas from the respiratory gas supply, through the nasal cannula, thence to the tip and, through the tip, into the patient's nostril, the tip including a first open end adapted for insertion into the patient's nostril and characterized by a first inside diameter; and
a second open end characterized by a second inside diameter that is greater than the inside diameter of the first end, the second end coupled to the nasal cannula for coupling to a respiratory gas supply.

6. The apparatus of claim 5 wherein the nasal cannula is a flexible nasal cannula having an inside diameter of between eight and thirty millimeters.

7. The apparatus of claim 5 further comprising:
an end-segment coupled to the nasal cannula, the end-segment including the tip.

8. The apparatus of claim 7 wherein the end-segment includes first and second open ends coupled to the cannula to convey respiratory gas to a nostril tip located along the end-segment between the first and second open ends.

9. The apparatus of claim 7 wherein the end-segment includes first and second ends, the first end coupled to the cannula to convey respiratory gas to a nostril tip located proximate the second end.

10. The apparatus of claim 5 wherein the inside diameter of the tip varies linearly from a greatest girth at its cannula-end to a minimal girth at its nostril-end.

11. The apparatus of claim 5 wherein the inside diameter of the tip varies non-linearly from a greatest girth at its cannula-end to a minimal girth at its nostril end.

12. The apparatus of claim 11 wherein the tip includes a conical and a cylindrical segment.

13. The apparatus of claim 5 wherein the inside diameter of the narrowest portion of the tip lies in a range between 2.5 and 7.0 millimeters.

14. The apparatus of claim 5 wherein the inside diameter of the widest portion of the tip is no greater than the diameter of the cannula from which it emerges.

15. The apparatus of claim 5 wherein the nostril end of the tip is non-occluding when inserted in a nostril.

16. The apparatus of claim 5 including two tips spaced-apart a distance to accommodate insertion of each tip into each of a patient's nostrils.

17. The apparatus of claim 9 further comprising an element for supporting the cannula in place with a patient.

18. The apparatus of claim 5 wherein the tip extends from a end-segment a distance of between eight and twenty-five millimeters.

19. The apparatus of claim 16 wherein the center-to-center distance between the two tips falls in a range between ten and forty millimeters.

20. An apparatus, comprising:
a heated-plate respiratory gas humidifier;
a high flow rate respiratory gas compressor; and
a high flow respiratory gas conduit, the compressor, and humidifier combined to supply humidified respiratory gas through the conduit at a high rate of flow to a patient, wherein the conduit includes tips for insertion into a patient's nostrils for open delivery of respiratory gas, the conduit terminates substantially at the nostril-insertion end and the tips include a first open end characterized by a first inside diameter, the first end adapted for insertion into the patient's nostril; and
a second end characterized by a second inside diameter that is greater than the inside diameter of the first end, the second end adapted for mating engagement with a nasal cannula.
